(19) United States
(12) Patent Application Publication
(10) Pub. No.: US 2016/0265016 A1
(43) Pub. Date: Sep. 15, 2016

(54) ARCHAEA AND LIPID COMPOSITIONS OBTAINED THEREFROM

(71) Applicant: K+S AKTIENGESELLSCHAFT, Kassel (DE)

(72) Inventors: Dirk ZIMMERMANN, Karben (DE); Heiko PATZELT, Wolfenbuettel (DE); Elke MAJEWSKI, Kassel (DE); Joerg-Heino SACHSE, Kassel (DE); Christine WEHR, Mittenaar (DE)

(73) Assignee: K+S AKTIENGESELLSCHAFT, Kassel (DE)

(21) Appl. No.: 15/161,354

(22) Filed: May 23, 2016

Related U.S. Application Data
(62) Division of application No. 13/997,099, filed on Oct. 24, 2013.

(30) Foreign Application Priority Data
Feb. 17, 2010 (DE) 102010008353.4

(51) Int. Cl. C12P 19/44; A61K 31/7028; C12P 7/64; A61K 9/127; A61K 35/66; C12P 9/00; A61K 31/661

(52) U.S. Cl. CPC C12P 19/44; C12P 9/00; A61K 31/7028; A61K 31/661; A61K 9/127; A61K 35/66; C12P 7/6463; C12P 7/6481

(57) ABSTRACT

This invention concerns microorganisms, to be precise Archaea, which, in case of cultivation at 25° C., indicate unsaturated ether lipids in quantities of at least 10% with reference to the total amount of ether lipids. In a further aspect, this invention is directed towards microorganisms within the Archaea, in particular from those of the class halomebacteria, in particular the order of Halobacteriales, in particular the family Halobacteriaceae, in particular the genus Haloarcula and Haloferax containing lipid compositions. These lipid compositions, in particular liposomes, are characterized by the presence of large quantities of unsaturated ether lipids. In a further aspect, the existing invention concerns a process for the extraction of these unsaturated ether lipids from the designated Archaea.

This is a divisional of application Ser. No. 13/997,099, filed Oct. 24, 2013, which is the National Stage of International application no. PCT/DE2011/000141, filed Feb. 16, 2011, which claimed priority to German patent application no. 102010008353.4, filed Feb. 17, 2010, of which all of the disclosures are incorporated herein by reference in their entireties.

The invention relates to microorganisms, particularly Archaea, which when cultured at 25° C. contain unsaturated ether lipids in amounts of at least 10% in relation to the total ether lipids. The invention further relates to certain lipid compositions obtainable from Archaea, particularly (within the Archaea) from the class of Halomebacteria, the order of Halobacteriales, the family of Halobacteriaceae, and in particular from the genuses Haloarcula and Haloferax. These lipid compositions, particularly liposomes, are characterized by the presence of large amounts of unsaturated ether lipids. The invention still further relates to a method of obtaining such unsaturated ether lipids from the described Archaea.

### STATE OF THE ART

Archaea, sometimes also called “archaebacteria”, are one of the three kingdoms (“domains”) of unicellular organisms (the others being the Bacteria and the Eukaryota). Archaea are single-cell organisms with a usually closed DNA molecule. They differ from Bacteria in their sequence of ribosomal 16S RNA, and also by other genetic physiological, structural, and particularly also biochemical characteristics.

Many species of Archaea are adapted to extreme environmental conditions. The Archaea include thermophilic microorganisms, acidophiles, and alkalophiles.

Although Archaea in some respects have substantial similarity to other prokaryotes, namely the Bacteria, nonetheless they have numerous distinctive characteristics. One of these, e.g., consists in the structure of the cell wall of the Archaea. It is comprised of a “bilayer” formed from phospholipids and glyco-lipids, and a crystalline surface layer above the bilayer (referred to as the “surface layer” or “S-layer”). In contrast to Bacteria, Archaea do not contain any murein (peptoglycan), and may have substantial multiplicity in the structure of their bilayers. Also, the composition of the plasma membrane in Archaea is distinctive. In Bacteria and Eukatyota, fatty acids are bound to glycerin via an ester bond. In Archaea a bilayer membrane constructed from glycerin diethers is found, and fatty alcohols derived from isoprene units are found in their hydro-phobic chains, instead of simple fatty acids. Also noteworthy are the trans-membrane lipids which are formed from double-long chains which are ethered on both sides of the membrane with glycerin, to form diglyceride tetra-ethers, which further stabilize the membrane. Thus, e.g., hyperthermophilic Archaea often have such stabilized membranes with diglyceride tetra-ethers. In halo-philic Archaea, the S-Layer has a shape-stabilizing function on its inner side. All of the membrane lipids are typically derivatives of a C₂₀-C₂₀-dialkylglyceryl di-ether, namely sn-2,3-diphytanylglyceryl diether (so-called “archaeol”).

Phospholipids of halophilic Archaea contain as their main component phosphatidylglyceryl phosphate methyl ester (PGP-ME), phosphatidylglyceryl phosphate (PGP), and as components in typically smaller proportions phosphatidylglycerin (PG), phosphatidylglyceryl sulfate (PGS), and phosphatidic acid (PA). The glycolipids contain principally diglycosyl archaeol, which may be singly or doubly sulfated, sulfated triglycosyl archaeol (S-TGA), and triglycosyl archaeol (TGA), as well as sulfated tetraglycosyl archaeol (S-TeGA). The sugar units are typically comprised of the hexoses glucose, mannose, or galactose. The galactose groups may also be present as furanose or pyranose.

Archaea, their components, and their applications, have been described to some extent in prior publications. E.g., WO93/08202 discloses the formation of stable liposomes from lipid extracts of Archaea. In that publication, novel ether lipids are described, which have been isolated from methanogenic and extremely halophilic species of Archaea; these include, e.g., saturated ether lipids, particularly derivatives of C₂₀-C₂₀-dialkylglyceryl diethers. Also described in that document are liposomes from Archaea, particularly liposomes which comprise the entire extract of the polar lipids from methanogenic and [(respectively)] halogenic Archaea. Areas of application stated to exist for such liposomes include: use as a research tool; use as an adjuvant or support for sub-stances, insecticides, genetic materials, and enzymes; and use as a cosmetic material.

Cosmetic preparates which contain inactivated cells or cell envelopes of halophiles or halotolerant microorganisms are disclosed in WO2004/103332. These cosmetic preparates have cell envelopes, or inactivated cells, which are obtained from the biomass or from an extract, and are particularly obtained from Archaea of the genus [sic] Halobacterium or from Bacteria of the genuses Halobacillus, Micrococcus, or Salinococcus. The inactivated cells or cell envelopes described there can also have pharmaceutical effects, such as protection against inflammation reactions, stimulation of defense mechanisms, etc.

Methods of characterizing ether lipids, such as glycolipids, are described e.g. in Qiu et al., Rapid Commun. Mass Spectrom., 2000, 14: 1586-1591. In this connection, membrane phospholipids or glycolipids from halophilic Archaea are studied with HPLC/ES-MS. For this purpose, Halobacterium salinarium was cultured and the lipid components were analyzed with the aid of MS. The lipids contained phospholipids and glycolipids. E.g., a PGP-ME with a double bond in the phytanyl side chain, to form an unsaturated ether lipid, was described. However, this was present in only very small amounts compared to the saturated form. The same phenomenon is described in Gibson et al., Systematic and Applied Microbiology, 2005, 28: 19-26. In this document, the analysis of phospholipids from Halorubrum lacusprofundi is described. It turned out that in culturing at 25° C., the following were principally produced from the microorganisms: saturated PGs, PGS, PGP, and PGP-ME. In culturing at 12° C., essentially the same phospholipids and glycolipids were found. However, in culturing at 12° C., there were some amounts of unsaturated forms of these phospholipids and glycolipids, with up to 6 double bonds. Only trace amounts of these unsaturated forms of the phospholipids and glycolipids were found in microorganisms cultured at 25° C.

In the production of biomass and culturing of microorganisms, in general the rate of propagation is usually correlated with the culturing temperature, i.e. at lower temperatures the microorganisms propagate more slowly, and the biomass production is reduced. Therefore culturing at higher temperatures is desirable for production of biomass comprised of microorganisms with relevant properties. Archaea which produce higher amounts of unsaturated ether lipids at higher temperatures (e.g. room temperature) are not described in the state of the art.

### BRIEF DESCRIPTION OF THE INVENTION

In a first embodiment, according to the invention, Archaea are prepared which in culturing at 25° C. have unsaturated ether lipids in an amount of at least 10%, preferably at least 20%, compared to the total amount of ether lipids.

The Archaea are preferably representatives of the class of Halomebacteria, order of Halobacteriales, and family of Halobacteriaceae, particularly the genuses Haloarcula and Haloferax.

In particular, the archaeols in these Archaea are unsaturated archaeols with 4 or 6 double bonds in the hydrocarbon chains—the alkyl chains—which chains are ordinarily a C₂₀-C₂₀-dialkyl group.

In a second embodiment, the present invention relates to “lipid compositions”, such as liposomes or inactivated cells or cell envelopes, which contain unsaturated ether lipids in an amount of at least 10%, preferably at least 20%, compared to the total amount of ether lipids. Preferred are such “lipid compositions”, such as liposomes or inactivated cells or cell envelopes, obtained from the inventive Archaea, particularly [of the genuses] Haloarcula and Haloferax.

Finally, the present invention relates to a method of obtaining unsaturated ether lipids, comprising culturing of the inventive Archaea in a culture medium at a temperature of at least 20 ° C., preferably at least 25 ° C., in order to obtain biomass containing the described unsaturated ether lipids.

### BRIEF DESCRIPTION OF THE FIGURES

FIG. 1 illustrates the proportion of saturated and unsaturated lipids from the inventive Archaea in comparison to the strain DSM5036 which was mentioned in Gibson et al. For this strain, [only] traces of unsaturated lipids were reported in culturing at 25° C.

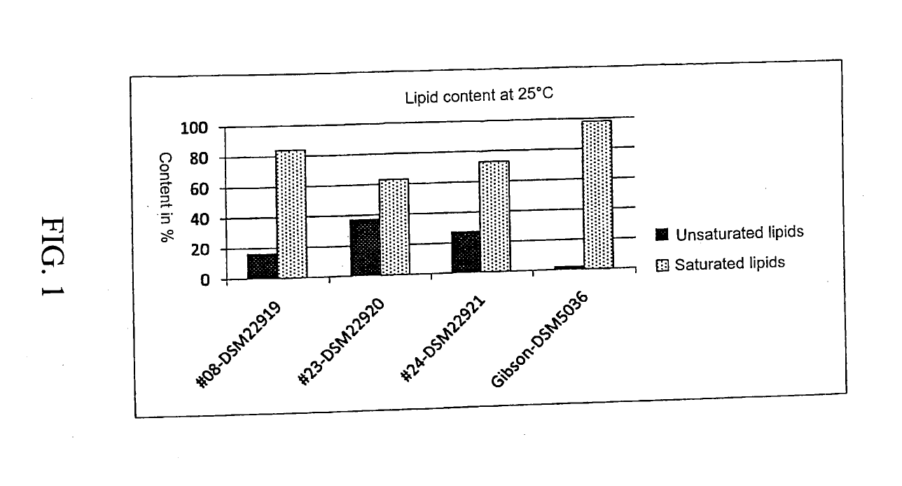

FIG. 2 illustrates an analysis of the ether lipids of an inventive strain, DSM22921.

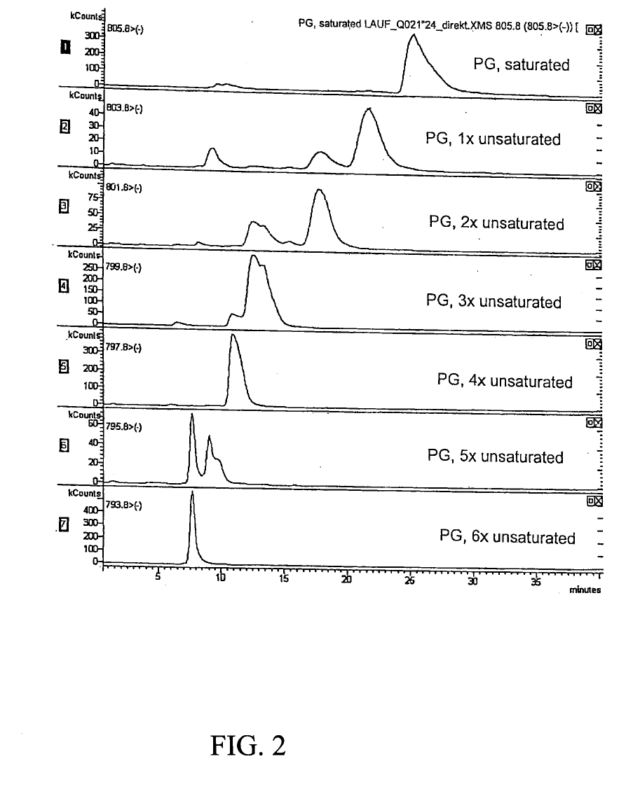

FIG. 3 illustrates a typical distribution of unsaturated and saturated PGs and saturated and unsaturated PGSs, in the inventive strains.

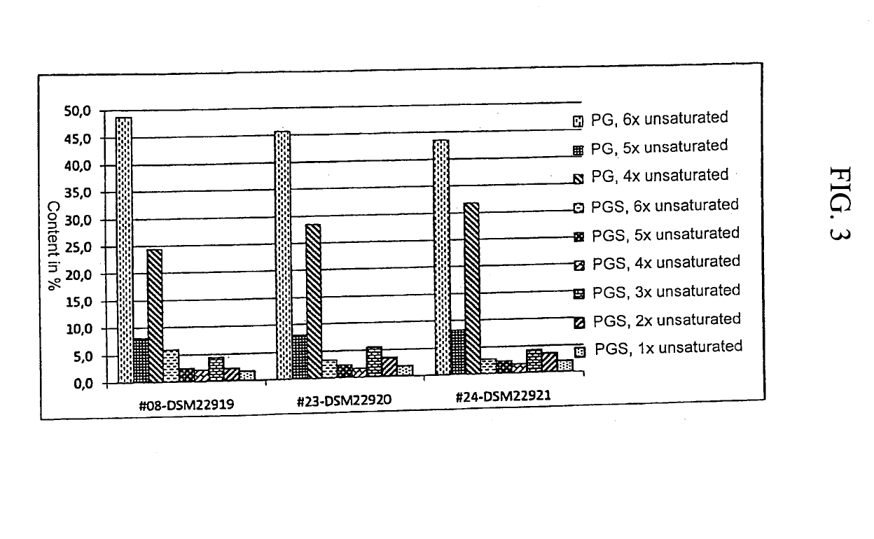

FIG. 4 illustrates the content of unsaturated lipids at different temperatures of inventive strains DSM22920, DSM22921, DSM22919, and DSM5036(Gibson).

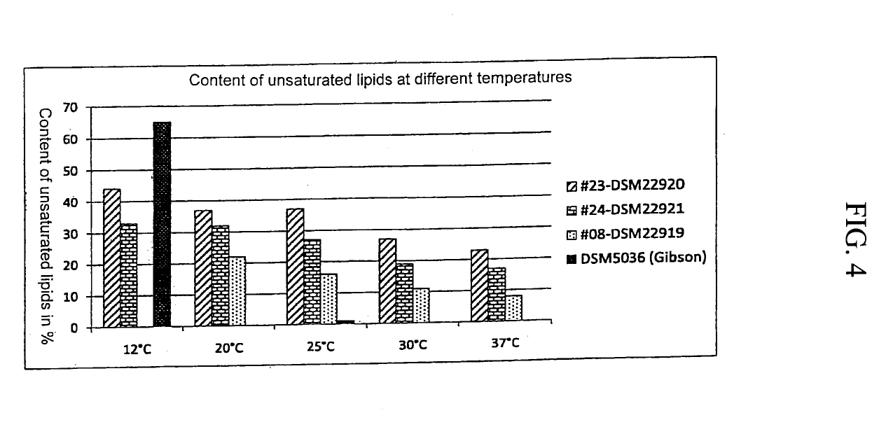

FIG. 5 illustrates the lipid content as a function of the fermentation time at 30° C. of inventive strains DSM22920, DSM22921, and DSM22919.

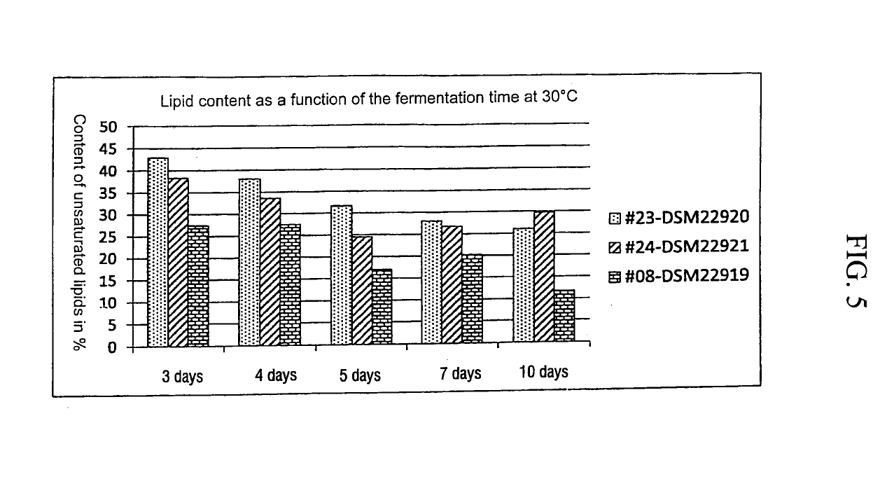

### DETAILED DESCRIPTION OF THE INVENTION

According to a first embodiment, according to the invention, Archaea are prepared which, in culturing at 25° C., have ether lipids in an amount of at least 10%, preferably at least 20%, compared to the total amount of ether lipids in the Archaea. According to the invention, for the first time (in history), Archaea are prepared which, under economic conditions, namely culturing at 25° C., or even 30° C., contain unsaturated lipids in an amount which enables commercial exploitation of these ether lipids in lipid compositions etc.

Previously described Archaea produced unsaturated ether lipids only in culturing at low temperature conditions, e.g. at 12° C. Under economically practicable conditions, with culturing temperatures of 20-25° C. or higher, only small amounts (traces) of these unsaturated ether lipids could be found. Commercial exploitation of these microorganisms to take advantage of the properties conferred by the unsaturated ether lipids is not possible.

In contrast, the inventive Archaea have large amounts of unsaturated ether lipids in relation to the total amount of ether lipids in the Archaea.

The amounts of unsaturated ether lipids are at least 10%, preferably at least 15-20%, particularly preferably 25-30%, compared to the total amount of ether lipids.

According to a preferred embodiment, the Archaea according to the invention are in the class of Halomebacteria, particularly the order of Halobacteriales, particularly the family of Halobacteriaceae. Members of this family include species of the genuses Halobacterium, Haladaptatus, Halalkalicoccus, Haloarcula, Halobaculum, Halobiforma, Halococcus, Haloferax, Halogeometricum, Halomicrobium, Halopiger, Haloplanus, Haloquadratum, Halorhabdus, Halorubrum, Halosimplex, Halostagnicola, Haloterrigena, Halovivax, Natrialba, Natrinema, Natronobacterium, Natronococcus, Natronolimnobius, Natronomonas and Natronorubrum.

Preferably the Archaea comprise those of the genus Haloarcula or Haloferax. Particularly preferred are members of the genus Haloarcula with the accession numbers DSM22919 and DSM22920 or members of the genus Haloferax with the accession number DSM22921.

I.e., a particularly preferred embodiment comprises Archaea with the characteristics of the strains with the above-stated accession numbers, having unsaturated ether lipids in the amount of at least 20% in relation to the total amount of ether lipids.

Preferably, the described ether lipids are those of general formula I

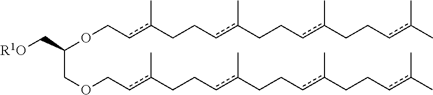

wherein

the hydrocarbon chains have a total of 1 to 8 double bonds ( [symbol] ), and may be substituted; and

R¹ is a sugar-containing group, which may be substituted, or a phosphatidyl group

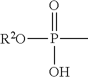

where

R² is hydrogen or a glycerin group, which glycerin group may be substituted, preferably substituted with a sulfatidyl group or a phosphatidyl group (but may be substituted with an alkyl chain).

These unsaturated ether lipids have the C_{20—}C₂₀-dialkyl groups in their hydrocarbon chains, with a total of 1-8 double bonds, preferably 1, 2, 3, 4, 5, or 6 double bonds. Thus in a singly unsaturated ether lipid of general formula I wherein the alkyl chains have a single double bond, one of the C₂₀-alkyl chains is singly unsaturated and the second alkyl chain is a saturated hydrocarbon chain. The positions of the double bonds are, e.g., at C(2), C(6), C(10), or C(14), on one or both alkyl chains. Possible positions of the double bonds are indicated in, inter alia, Gibson et al. The ether lipids are particularly preferably phospholipids, e.g. phosphatidic acid (PA). Other preferred ether lipids which are present in unsaturated form in the inventive Archaea are phosphatidylglycerin (PG) (also known as phosphatidylglycerol), phosphatidyl glycerin phosphate (PGP), and phosphatidyl glycerin sulfate (PGS).

These compounds are present primarily in saturated form but also to some extent in unsaturated form in the inventive Archaea. These compounds may have singly or multiply unsaturated side chains, e.g. singly, doubly, triply, quadruply, quintuply, sextuply, septuply, or octuply unsaturated side chains. Further, the phospholipids may be present in the form of dimers, as cardiolipin.

The ether lipids may also be present as unsaturated glycolipids. These glycolipids may include, in particular, unsaturated glycolipids of the group monoglycosyl archaeol (MGA), diglycosyl archaeol (DGA), DGA sulfate ester (S-DGA), DGA disulfate ester (S2-DGA), triglycosyl archaeol (TGA), TGA sulfate ester (S-TGA), tetraglycosyl archaeol sulfate ester (S-TeGA), and glycocardiolipins.

In a preferred embodiment, at least a part of the unsaturated ether lipids are PGs (phosphatidyl glycerins).

Although the proportion of unsaturated lipids in relation to the total amount of lipids has been found to decrease with increasing temperature, according to the invention it has been found possible to increase the absolute amount of unsaturated lipids even when the fermentation temperature is increased from 12° C. to 20° C., 25° C., or even 30° C., because the total yield of lipids at 25° C. and 30° C. is substantially greater than at 12° C. or 20 ° C. Therefore it is possible, on a scale which is acceptable, particularly economically acceptable, to obtain ether lipids and lipid compositions, such as liposomes [, with appreciable unsaturated ether lipids].

According to the invention, also cell envelopes or inactivated cells in the form of a pure biomass or in the form of extracts can be prepared. This means that presently using known methods one can prepare inactivated cells as biomass which cells are obtained directly from the fermentation. Alternatively, cell envelopes in the form of biomass or in the form of extracts can be prepared. Methods of analogous preparation of cell envelopes from the inventive Archaea are known to one skilled in the art.

According to another embodiment, the invention relates to lipid compositions, particularly liposomes, which can be obtained from the inventive Archaea. These lipid compositions, particularly liposomes (but also the abovementioned cell envelopes or inactivated cells) are distinguished in that they contain unsaturated ether lipids in an amount of at least 10% in relation to the total amount of ether lipids. Preferably, the compositions, inactivated cells, and cell envelopes contain unsaturated ether lipids in a proportion of at least 15-20%, preferably at least 25-30%, in relation to the total amount of ether lipids.

The inventive lipid compositions, particularly liposomes (or cell envelopes or inactivated cells) containing unsaturated ether lipids, are preferably obtainable from the microorganisms Haloarcula sp. with the accession number DSM22919 or DSM22920, or from Haloferax sp. with the accession number DSM22921.

The inventive lipid compositions, particularly the liposomes (or the cell envelopes or inactivated cells) are distinguished in that they contain large proportions of unsaturated ether lipids of [sic] the compounds described herein, in a proportion of at least 10%, preferably 20%, in relation to the total amount of ether lipids. This opens up the possibility of exploiting the advantageous properties of the unsaturated ether lipids. The spectrum of possible uses includes the known possible uses for ether lipids, e.g. as described in WO2004/103332 or WO93/08202.

According to another embodiment, the invention relates to a method of obtaining unsaturated ether lipids, particularly ether lipids according to general formula I

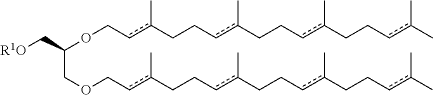

wherein

the hydrocarbon chains have a total of 1 to 8 double bonds ( [symbol] ), and may be substituted; and

R¹ is a sugar-containing group, which may be substituted, or a phosphatidyl group

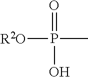

where

R² is hydrogen or a glycerin group, which glycerin group may be substituted, preferably substituted with a sulfatidyl group or a phosphatidyl group (but may be substituted with an alkyl chain);

wherewith the method comprises the step of culturing the inventive Archaea in a culture medium at a temperature of at least 20° C., preferably at least 25° C.

Preferably the inventive method employs Archaea according to the present invention, particularly Archaea from genuses such as Haloarcula and Haloferax. Particularly preferred for obtaining the described unsaturated ether lipids are the strains DSM22919, DSM22920 and DSM22921.

The inventive method is distinguished in that the culturing of the Archaea occurs at a temperature of at least 20° C., preferably at least 25° C. The culturing temperature may be 30° C. or higher, e.g. 37° C. or higher. After the culturing the cultured Archaea have an amount of unsaturated ether lipids of at least 10-15%, preferably at least 20-25% or even 30%, in relation to the total amount of ether lipids in the Archaea.

The inventive method is further distinguished in that in the culturing of the Archaea the “lag” phase is bridged over by addition of not less than 5% (vol/vol) of inoculum. The inoculum may be produced at a higher temperature than the culturing temperature (e.g. it may be produced at 30° C. or higher, preferably 37° C.), whereas the culturing is carried out at the inventive lower temperatures.

It was found, surprisingly [, in connection with the invention], that the yield of unsaturated lipids at the end of the exponential growth phase—after 3-4 days' duration of the fermentation—is higher than if one extends the fermentation until the steady-state phase is reached (which would correspond to the maximum biomass). Accordingly, in the inventive method the culturing of the Archaea is preferably carried out up to the end of the exponential growth phase, e.g. with a fermentation duration of 3-4 days at 25° C. Thus it is preferred to harvest the biomass before the steady-state phase is reached.

To recover the unsaturated ether lipids the inventive method also is comprised of the step of extraction of the biomass with an organic solvent, to separate out the lipids.

In this extraction, chloroform may be used, alone or mixed with, e.g., methanol and possibly water. To achieve good separation of the phases, a mixture of chloroform and water is preferred. According to a preferred embodiment, thereafter one separates the lipids using, e.g., a kieselgel column. In this step the column may be pre-conditioned with, e.g., chloroform, in order to separate out other components of the fraction obtained after the organic extraction, e.g. colorants. The glycolipids fraction is preferably eluted from the kieselgel column with the aid of, e.g., acetone. Then the phospholipids fraction can be eluted from the kieselgel column with, e.g., an alcohol, such as methanol. Appropriate techniques are known to one skilled in the art.

The unsaturated ether lipids may be separated with the aid of known techniques. E.g., one might mention chromatographic methods.

The unsaturated ether lipids may be separated in some detail or may be obtained in fractions containing various unsaturated ether lipids.

The method of extraction of lipids from the cell mass of Archaea, particularly the halophilic Archaea, and determination using LC-MS, is based on modifications of the method described in Gibson et al.

The inventive method allows preparative recovery of unsaturated ether lipids from the inventive Archaea.

The preparation of the inventive Archaea allows one to obtain unsaturated ether lipids or lipid compositions including cell envelopes and inactivated cells (which contain the unsaturated ether lipids) in amounts of at least 10% in relation to the total amount of ether lipids. It was found, surprisingly, [in connection with the invention], that in contrast to the embodiments in, e.g., Gibson et. al., unsaturated ether lipids can be obtained in substantial amounts even with culturing conditions of at least 20° C., preferably at least 25° C. This makes possible commercial exploitation of these unsaturated ether lipids.

The inventive (italics) Archaea (italics) are particularly those from the group of halophilic (italics) Archaea (italics) with the characteristics of the deposited strains DSM 22919, DSM 22920, and/or DSM 22921.

Hereinbelow the invention will be described in more detail with the aid of exemplary embodiments, which do not limit the scope of the invention.

### Source and Cultivation Conditions of the Archaea

The three inventive Archaea strains DSM22919, DSM22920 and DSM22921 were extracted from saline-content locations of the Sigmundshall Works of K+S Kali GmbH. DSM5036 was obtained from DSMZ GmbH, Braunschweig.

The cultivation of the halophilic Archaea, which was employed in the example for the manufacture of unsaturated ether lipids, was carried out as follows:

Nutrient medium DSMZ No. 372 (modified).

| | | |
|---|---|---|
| Yeast extract | 5 | g |
| Casein hydrolyzate | 5 | g |
| Sodium-L-glutamate | 1 | g |
| monohydrate | | |
| Tri-sodium citrate | 3 | g |
| NaCl | 200 | g |
| KCl | 2 | g |
| MgSO₄•7 H₂O | 20 | g |
| FeCl₂•6 H₂O | 36 | mg |
| Manganese chloride | 1 | ml |
| strain solution | | |
| Extran ® AP31 | 200 | μl |
| (anti-foaming agent) | | |
| Deionized water | ad. 1000 | ml |
| Manganese chloride | | |
| strain solution | | |
| MnCl₂•4 H₂O | 36 | mg |
| Deionized water | ad. 100 | ml |

The pH-value of the nutrient medium was adjusted with KOH from 7.0 to 7.2, and the medium was autoclaved for 20 min at 121° C.

The culture was inoculated under sterile conditions in 25 ml nutrient medium, and incubated in a 100 ml Erlenmeyer flask on a circular shaker at approx. 120 rpm at 37° C. for 7 days. Then the culture was transferred into 225 ml nutrient medium and incubated for a further 7 days at 37° C. at 120 rpm on a circular agitator. The inoculum obtained was transferred to a 4,750 ml nutrient medium with the above-designated composition and incubated with a magnetic stirrer with 650 rpm at 25° C. for a further 4 days. The culture was cleaned with approximately 3.8 l/min water and moist room-air vented over a diaphragm pump by means of a sterile silicone-ring hose ventilation unit. The biomass obtained was separated from the nutrient medium through centrifuging (7,000 rpm 6,566 g) for 20 minutes. The cell pellet was washed with 20 ml basal salt washing solution, and centrifuged again at 6,566 g for 20 minutes. Where appropriate, the biomass pellet obtained was then frozen to −80° C. for further processing.

### Basal Salt Washing Solution

| | | |
|---|---|---|
| NaCl | 200 | g |
| KCl | 2 | g |
| MgSO₄•7 H₂O | 20 | g |
| Deionized water | ad. 1000 | ml |

### Extraction

A volume of 120 ml extraction solution (CHCl₃/MeOH/H2O, 5/10/4, v/v/v) is added to a cell pellet of approx. 30 g mass in a 250 ml ballast bottle, and the bottle closed off. After strong shaking until complete re-suspension of the cell pellet, the ballast bottle is placed into a cooled ultrasonic bath for 15 min. With a measuring cylinder, 32 ml chloroform and 32 ml HPLC water are added. The ballast bottle is closed off again and shaken vigorously. In this step, a 3-phase system is formed after centrifuging (chloroform phase/cell components/dilute phase).

After centrifuging at approx. 2,500 rpm (approx. 1,900 g) for 10 min at approx. 4° C., the lower phase (approx. 60 ml chloroform) is extracted with a disposable syringe with long stainless-steel cannula and transferred into a 100 ml ballast bottle. A spoonful of dry sodium salt is added to the isolated chloroform phase, the bottle closed off and strongly shaken.

After centrifuging at approx. 3,000 rpm for 5 min at 4° C., the liquid remaining is decanted and the residue extracted with a Pasteur pipette and transferred into a 100 ml round-bottom flask. The chloroform is concentrated at 30° C. in vacuum to approx. ⅓ of the output volume.

### Cleaning Off the Raw Extract by Means of Silica Gel Column

A solid-phase column “BAKERBOND Silica Gel” (5 g column bed) is clamped in a mount bracket and pre-conditioned with chloroform. To that is added 20 ml chloroform with a pipette and this is allowed to flow through without over-pressure or under-pressure.

### Manufacture of the Chloroform Fraction for the Extraction of Dyes

The chloroform extract as obtained above (approx. 20 ml; up to 100 ml can be added to the column) is added to the solid-phase column. The extract now percolates slowly, without over-pressure or under-pressure. A brown-glass bottle is placed under the column and 20 ml chloroform are added to the column for elution. After the entire solvent has run through, approx. 50 ml air is pressed slowly through the column with a disposable syringe (and column adapter), in order to flush out chloroform residue still in the brown-glass bottle.

### Manufacture of the Acetone Fraction for the Extraction of Glycolipids

A further brown-glass bottle is placed under the column and 20 ml acetone is added to the column. When the entire elution agent has run through, approx. 50 ml air is pressed slowly through the column with a disposable syringe (and column adapter) in order to flush out acetone residue still in the brown-glass bottle.

### Manufacture of the Methanol Fraction for the Extraction of Phosphatides

A further brown-glass bottle is placed under the column and 90 ml methanol added to the column. When the entire elution agent has run through, approx.

50 ml air is pressed slowly through the column with a disposable syringe (and column adapter) in order to flush out methanol residue still in the brown-glass bottle.

### Measurement

The measurement of the lipid fractions is implemented by means of LC-ESI-MS.

### Chromotography Conditions

System: Varian 320-MS
Pre-column: RP18
Separation column: Varian Polaris RP18, 150×2 mm, 3 μm
Temp. column: 30° C.
Mobile liquid: Methanol/water/NH₄-Ac, 94/4/2 (v/v/v)
Gradient: Without, isocratic
Flow: 0.3 ml/min
Detection: Mass spectrometer
Injection volume: 5-20 μl (μl pickup method)
Rum time: 60 min

### Mass-Spectrometer Detection

Ion source: ESI
Scan mode: Centroid
SIM width: 0.700 amu total
Detector: EDR
   Turn off source at the end of run
Segments: 1
CID Gas: Off
Scan time requested: 1,520 Sec.
Peak width selection: Q1 Peak width 1.00
   Q3 Peak width 1.50
ESI needle voltage negative: −4,500.00
ESI shield voltage negative: −600.00
Drying gas temperature: 300.00
API housing temperature: 50.00
Nebulizer gas pressure: 55.00
Drying gas pressure: 18.00
6 Port valve: “MS”
Data collection: ON

In Illustration 2 is shown a typical result of the analysis of the ether lipids, here the PG, in the inventive strain DSM22921. All forms of unsaturated PG with one to six double bonds can be verified.

In Illustration 1 is represented the lipid content of saturated and unsaturated lipids in the inventive Archaea and the DSM5036 strain as described in Gibson et al. As can be clearly identified, the content of unsaturated lipids is considerably higher with the inventive strains than in the known Archaea strain.

More precise analysis of the unsaturated ether lipids for PG and PGS is represented in Illustration 3. As shown, the unsaturated forms indicated can be verified in all listed strains.

In Illustration 4 are represented the contents of the unsaturated ether lipids after manufacture of the respective biomass with different temperatures. It can be clearly identified that, also with higher cultivation temperatures, the inventive Archaea manufacture unsaturated phosphatides in large quantities.

Furthermore, investigations of the dependence on the fermentation time were carried out. The cultivation was implemented, as described above, with different duration. At different times, biomass was harvested with the above described processes and analyzed for its lipid content. The results are represented in Illustration 5. It can be identified that the content of unsaturated ether lipids in the growth stage (day 3 to day 4) exceeds the content of unsaturated ether lipids in the steady-state phase. Therefore the harvest of the biomass is preferably carried out before the beginning of the steady-state phase.

| | 12° C. | 20° C. | 30° C. | 37° C. |
|---|---|---|---|---|
| #24 Content | 37.2 | 41.6 | 17.5 | 22.6 |
| unsaturated | | | | |
| in % | | | | |
| #23 Content | 47.7 | 37.7 | 29.1 | 25.6 |
| unsaturated | | | | |
| in % | | | | |
| #8 Content | No value | 27.5 | 23.9 | 11.3 |
| unsaturated | | | | |
| in % | | | | |

1. A lipid composition comprising the total extract of ether lipids obtained from archaea cultivated at 25° C., wherein the ether lipids comprise at least 10% of unsaturated ether lipids based on the total amount of the ether lipids.

2. The lipid composition of claim 1, wherein the unsaturated ether lipids are obtainable from a halophilic microorganism with the accession number DSM22919, DSM22920 and DSM22921.

3. A process for extraction of unsaturated ether lipids of the formula I: 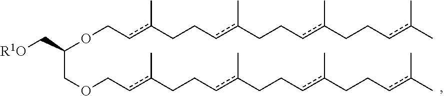
wherein the hydrocarbon chains have a total of one to eight double bonds ( [symbol] ) and are optionally substituted,
R′ is a sugar-containing residue which can be substituted or a phosphatidyl group 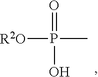
wherein R² is hydrogen or a glycerin residue, wherein the glycerin residue is optionally substituted the process comprising cultivating of Archaea in a culture medium at a temperature of at least 20° C., thereby obtaining a biomass comprising ether lipids containing the unsaturated ether lipids.

4. The process of claim 3, comprising harvesting the biomass before reaching a steady-state phase.

5. A process of claim 3, comprising adding at least 5% (v/v) of an inoculum to the culture medium.

6. The process of claim 5, wherein the inoculum was cultivated at a temperature of at least 30° C.

7. The process of claim 3, a strain selected from the group consisting of DSM22919, DSM22920 and DSM 22921 is cultivated.

8. The process of claim 3, further comprising extracting the biomass with an organic solvent for the separation of the ether lipids.

9. The process of claim 3, further comprising isolating the ether lipids by using a silica gel column.

10. The process of claim 3, further comprising cleaning or concentrating the ether lipids by using HPLC.

11. The process of claim 3, wherein the cultivating of Archaea is conducted at a temperature of at least 25° C.

12. The process of claim 3, wherein the glycerin residue is substituted with at least one member selected form the group consisting of a sulfatidyl group, a phosphatidyl group, and an alkyl chain.

13. The lipid composition of claim 1, having a form of stable liposomes formed from the ether lipid extract of Archaea.

14. The lipid composition of claim 1, wherein the ether lipids comprise at least 20% of unsaturated ether lipids based on the total amount of the ether lipids.

15. The lipid composition of claim 1, wherein the ether lipids comprise from 15% to 20% of unsaturated ether lipids based on the total amount of the ether lipids.

16. The lipid composition of claim 1, wherein the ether lipids comprise from 25% to 30% of unsaturated ether lipids based on the total amount of the ether lipids.

17. A pharmaceutical composition comprising inactivated cells or cell envelopes of Archaea, and at least one pharmaceutically acceptable excipient, wherein the cell envelopes or inactivated cells are obtained from a biomass or an extract obtained from Archaea.
